(19) United States
(12) Patent Application Publication
(10) Pub. No.: US 2009/0043311 A1
(43) Pub. Date: Feb. 12, 2009

(54) Offset distraction device and method of use

(76) Inventors: Tibor B. Koros, Moorpark, CA (US); Gabriel J. Koros, Moorpark, CA (US)

Correspondence Address: ARTHUR FREILICH;FREILICH, HORNBAKER & ROSEN, 20555 DEVONSHIRE ST. #372, CHATSWORTH, CA, 91311, US

(21) Appl. No.: 12/082,285

(22) Filed: Apr. 9, 2008

Related U.S. Application Data
(63) Continuation-in-part of application No. 29/289,952, filed on Aug. 7, 2007.

(51) Int. Cl. A61B 17/58; A61B 17/56

(52) U.S. Cl. 606/90; 606/301; 606/96

(57) ABSTRACT

A distraction device employing a bone fastener and an axially displaced post to provide enhanced access to a surgical site. The distraction device is comprised of a rigid arm or link having a bone fastener, e.g., screw, extending from a first end and a post extending oppositely to the fastener, from a second end. The bone fastener is configured so that when anchored to a patient's bone, the rigid arm is able to pivot around the fastener to enable a surgeon to position the post for optimal access.

### RELATED APPLICATIONS

This application is a CIP of U.S. application Ser. No. 29/289,952 filed Aug. 7, 2007 whose disclosure is by reference incorporated herein.

### FIELD OF THE INVENTION

This invention relates to distraction devices useful in a variety of medical procedures such as cervical spinal distraction and spinal fusion and more particularly to a distraction device and method of use for facilitating access to a surgical site.

### BACKGROUND OF THE INVENTION

Distraction devices, or distraction screws, are widely used in various types of spinal surgical procedures for distracting or forcibly separating, bone segments, e.g., vertebrae. Typically such devices include a screw having an external thread at its distal end configured to screw into bone and an elongate post at its proximal end axially aligned with the screw. A typical surgical procedure employs two distraction devices with the respective screws being threaded into neighboring bone segments and with the respective posts projecting away from the bone segments substantially parallel to one another. A surgeon can then couple a retractor instrument to the posts and operate it to apply a displacement force to the posts to move the bone segments in a desired manner.

Such surgical procedures must be very carefully performed to achieve optimum results. Particularly, the surgeon must accurately place the screws and the posts so that they produce the intended effect on the bone segments in response to the applied displacement force. Oftentimes the surgeon may not have adequate access to optimally place the screws in the bone segments and optimally position the posts for mating with the retractor instrument.

### SUMMARY OF THE INVENTION

The present invention relates to a distraction device employing a bone fastener and an offset, i.e., axially displaced post to provide enhanced access to a surgical site.

A distraction device in accordance with the invention is comprised of a rigid arm or link having a bone fastener, e.g., screw, extending from a first end and a post extending oppositely to the fastener, from a second end.

A preferred bone fastener is mounted so that when anchored to a patient's bone, the rigid arm is able to pivot around the fastener to enable the surgeon to position the post for optimal access by the surgeon, e.g., for coupling to a retractor instrument.

In accordance with a preferred embodiment, the bone fastener comprises a screw which is held captive in a hole formed through a first end of the rigid arm. Also, the underside of the rigid arm is preferably relieved to facilitate pivoting around the bone fastener.

### BRIEF DESCRIPTION OF THE FIGURES

FIG. 1 is a schematic isometric view of first and second distraction devices in accordance with the invention being installed on a patient's spine;

FIG. 2 is a sectional view of first and second distraction devices in accordance with the invention installed on first and second bone segments;

FIG. 3 is a sectional view similar to FIG. 2 but showing a lateral displacement force being applied to the device posts for moving said bone segments;

FIG. 4 is a top plan view of a preferred distraction device in accordance with the invention;

FIG. 5 is sectional view taken substantially along the plane 5-5 of FIG. 4;

FIG. 6 is an isometric view of the preferred distraction device of FIG. 5 showing how the rigid link and post can be pivoted around the bone screw; and

FIG. 7 is an exploded isometric view of the preferred distraction device of FIG. 6 showing how the bone screw and post are mounted on the rigid link.

### DETAILED DESCRIPTION

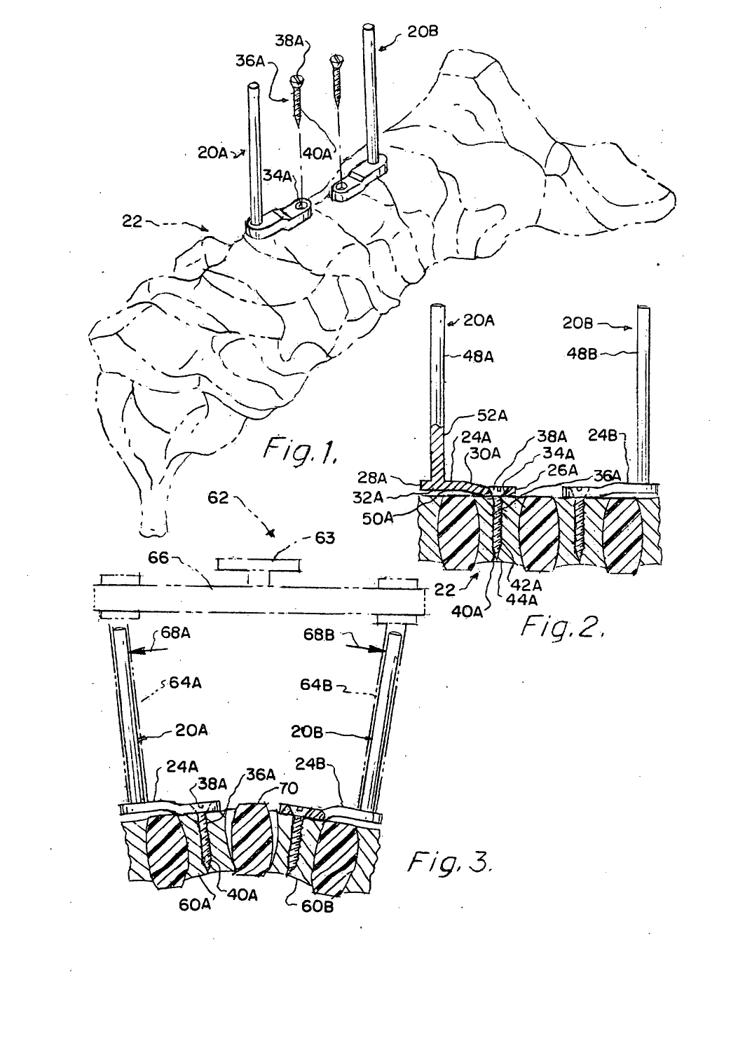

Attention is now directed to FIG. 1 which illustrates first and second distraction devices 20A, 20B, in accordance with the invention, ready for installation on a patient's spine 22. The devices 20A, 20B are preferably identical. Accordingly, the discussion herein will primarily focus on the structure and operation of device 20A but it should be understood that the discussion applies equally to device 20B unless otherwise stated.

Conventional distractions devices, or screws, are used in various medical procedures for moving bone segments, e.g., vertebrae, relative to one another. Such distraction devices typically comprise an elongate member having a distal screw end configured for threading into a patient's bone segment and an axially aligned proximal post end configured for coupling to a force applicator, e.g., retractor instrument. In typical use, two such devices are screwed into respective neighboring bone segments at an open surgical site. A surgeon then applies a displacement force between the respective device posts which act as levers to move/tilt the bone segments to which they are anchored. The present invention is directed to enhanced distraction devices designed to offer the surgeon improved access to the posts to facilitate optimum bone segment movement.

With continuing reference to FIGS. 1-3, the distraction device 20A is comprised of a rigid arm, or link, 24A having a first end portion 26A and a second end portion 28A. The arm 24A defines an upper surface 30A and a lower surface 32A. FIGS. 1-3 illustrate a hole 34A in the first end portion 26A extending therethrough from the upper surface 30A to the lower surface 32A. The hole 34A can be configured to accommodate the head of a bone fastener, e.g., bone screw, 36A. More particularly, 36A has a head 38A which can be tapered to seat in correspondingly shaped hole 34A and a body 40A having an external thread 42A configured for anchoring into a first bone segment 44A.

The arm 24A second end portion 28A carries an elongate post 48A which projects from the arm upper surface 30A in a direction substantially opposite to the projection of screw 36A. The arm 24A is configured to allow a surgeon to pivot, or swing, the arm around the screw 36A to position the post 48A in an appropriately accessible position. The arm lower surface 32A is preferably relieved at 50A to allow the arm 24A to pivot easily. The post 48A can be formed integral with the arm 24A or can be secured to the arm 24A by a variety of means, e.g., by threading the post lower end 52A into a threaded hole in the arm.

In typical use, distraction devices 24A and 24B will be installed in neighboring bone segments 60A, 60B, respectively, as shown in FIGS. 2, 3. The surgeon can then pivot the arms 24A, 24B to position the posts 48A, 48B in a position readily accessible for mounting a force applicator 62, e.g., retractor instrument 63, shown in phantom in FIG. 3. An exemplary force applicator includes hooks or sleeves 64A, 64B and a ratchet mechanism 66 for forcing the sleeves laterally, as represented by directional arrows 68A, 68B. Note in FIG. 3 that the force applicator 62 is able to force the posts 48A, 48B apart to correspondingly move/tilt the bone segments 60A, 60B. This procedure relieves compression on the intermediate disk material 70.

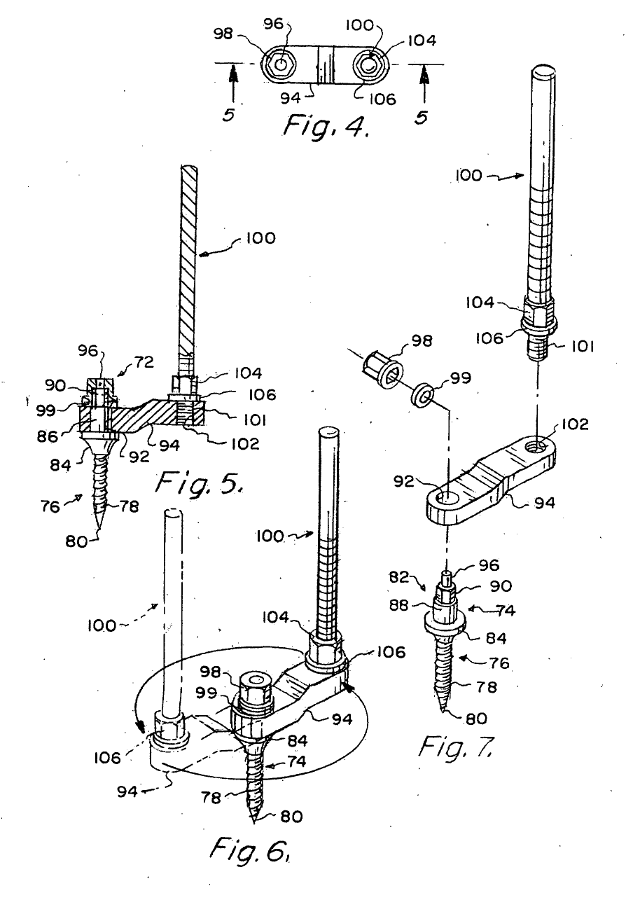

Attention is now directed to FIGS. 4-7 which illustrate the distractor device 24A modified to incorporate a preferred bone fastener 72. The bone fastener 72 is comprised of a body 74 having a lower portion 76 bearing a suitable bone thread 78. The body lower portion 76 terminates in a pointed end 80 to facilitate bone penetration. The body 74 has a upper end 82 including a radial flange 84, a cylindrical bearing 86, and a multifaceted, e.g., hexagonal, upper end 90. The cylindrical bearing 86 is intended to be accommodated in a cylindrical hole 92 extending between the upper and lower surfaces of rigid arm 94. The body 74 terminates at its upper end in a short stud 96 configured to receive a cap 98 which is intended to be press-fit thereon. The cap 98 retains the fastener 76 captive in the hole 92. A split washer 99 is preferably inserted on the stud 96 between the cap 98 and the area upper surface.

FIGS. 4-7 also illustrate an exemplary post 100 which is threaded at 101 proximate to its lower end for screwing into internally threaded hole 102 in rigid arm 94. The post 100 preferably carries a nut 104 having a flange 106 for bearing against the upper surface of arm 94 to stabilize the post 100. In use, the arm 94 can pivot around screw 72, as shown in FIG. 6, to selectively position the post 100.

The foregoing describes a distraction device and method of use which facilitates surgical site access when used in a variety of surgical procedures. Although only a limited number of exemplary embodiments have been described, it should be recognized that variations and modifications may readily occur to those skilled in the art which fall within the spirit of the invention and intended scope of the appended claims. For example only, although the preferred embodiments illustrated herein show both the fastener, e.g., 72, and post, e.g., 100, as projecting substantially perpendicularly from the arm 94, this relative orientation can be readily modified.

1. A distraction device comprising:
an arm having laterally spaced first and second end portions, said arm defining oppositely directed lower and upper surfaces;
an elongate bone fastener from said arm lower surface for attaching said arm first end portion to a patient's bone segment;
a post projecting from said arm upper surface second end portion; and wherein
said arm is mounted for pivotal movement around the axis of said elongate bone fastener.

2. The device of claim 1 wherein said arm first end portion includes a hole extending therethrough from said upper surface to said lower surface; and wherein
said fastener comprises a screw dimensioned to extend through said hole, said screw including an external thread configured to thread into a patient's bone.

3. The device of claim 2 including means for retaining said screw captive in said hole.

4. The device of claim 1 wherein said elongate bone fastener projects substantially perpendicularly from said arm.

5. The device of claim 4 wherein said post projects from said arm in a direction substantially opposite to the projection of said bone fastener.

6. A device for use in surgical procedures for the distraction of bone segments comprising:
a rigid elongate link having spaced first and second ends;
a bone screw projecting from said link first end for threading into a patient's bone segment;
a post projecting from said link second end in a direction substantially opposite to said projection of said bone screw; and wherein
said link is configured for pivotal movement around said bone screw.

7. The device of claim 6 wherein said bone screw is captive in said link.

8. The device of claim 6 wherein said bone screw projects from a lower surface of said link and wherein said post projects from an upper surface of said link; and wherein
said link lower surface is relieved to facilitate pivoting of said link around said screw.

9. The device of claim 6 wherein said post is externally threaded to facilitate coupling to a retractor sleeve.

10. The device of claim 6 wherein said screw projects substantially perpendicularly from said link.

11. A method of affixing first and second posts to respective first and second bone segments to facilitate performance of a distraction procedure, said method comprising:
mounting a first rigid link for pivotal movement around a first bone fastener configured for attaching to a first bone segment;
providing a first post on said first rigid link spaced from said first bone fastener and projecting in a direction opposite to the projection of said first bone fastener;
mounting a second rigid link for pivotal movement around a second bone fastener configured for attaching to a second bone segment;
providing a second post on said second rigid link spaced from said second bone fastener and projecting in a direction opposite to the projection of said second bone fastener; and
applying a lateral force to said first and second posts to move said first and second bone segments.
